SEQUENCE LISTING (1 sequence)

SEQ ID NO: 1 - protein, 24 aa, Artificial Sequence
  NS5A/B protease cleavage
ATVSEEASEDVVCCSMSYTWTGAL

(19) United States
(12) Patent Application Publication
(10) Pub. No.: US 2009/0203008 A1
(43) Pub. Date: Aug. 13, 2009

(54) Rapid method to determine inhibitor sensitivity of NS3/4A protease sequences cloned from clinical samples

(76) Inventors: Steven W. Ludmerer, North Wales, PA (US); Donald J. Graham, Green Lane, PA (US); David B. Olsen, Lansdale, PA (US)

Correspondence Address: MERCK AND CO., INC, P O BOX 2000, RAHWAY, NJ, 07065-0907, US

(21) Appl. No.: 12/308,010

(22) PCT Filed: Jun. 4, 2007
(86) PCT No.: PCT/US07/13154

§ 371 Date: Dec. 4, 2008

Related U.S. Application Data
(60) Provisional application No. 60/811,991, filed on Jun. 8, 2006.

(51) Int. Cl. C12Q 1/68

(52) U.S. Cl. 435/6

(57) ABSTRACT

A method for measuring HCV NS3/4A activity from a HCV NS3/4A sequence, comprising obtaining and cloning the sequence into a mammalian expression vector, transiently transfecting a mammalian cell with the vector, which includes a reporter construct encoding a HCV NS3/4A cleavage site fused to a detectable label, measuring signal production from the label resulting from cleavage at the cleavage site, and measuring effects on signal production by addition of a test compound.

### BACKGROUND OF THE INVENTION

The references cited in the present application are not admitted to be prior art to the claimed invention.

It is estimated that about 3% of the world's population are infected with the Hepatitis C virus (HCV). Annemarie Wasley and Miriam J. Alter, 20(1) SEMINARS IN LIVER DISEASE 1-16 (2000). Exposure to HCV results in an overt acute disease in a small percentage of cases, while in most instances the virus establishes a chronic infection causing liver inflammation and slowly progresses into liver failure and cirrhosis. Sten Iwarson, 14 FEMS MICROBIO. REV. 201-04 (1994). Epidemiological surveys indicate HCV plays an important role in hepatocellular carcinoma pathogenesis. Michael C. Kew, 14 FEMS MICROBIO. REV. 211-20 (1994); Harvey J. Alter, 85(7) BLOOD 1681-95 (1995).

The HCV genome consists of a single strand RNA about 9.5 kb in length, encoding a precursor polyprotein about 3000 amino acids. G. Kuo et al., 244 SCIENCE 362-64 (1989); Qui-Lim Choo, 244 SCIENCE 359-62 (1989); A. Takamizawa et al., 65(3) J. VIROLOGY 1105-13 (1991). The HCV polyprotein contains the viral proteins in the order: C-E1-E2-p7-NS2-NS3-NS4A-NS4B-NS5A-NS5B.

Individual viral proteins are produced by proteolysis of the HCV polyprotein. Host cell proteases release the putative structural proteins C, E1, E2, and p7, and create the N-terminus of NS2 at amino acid 810. Hiroto Mizushima et al., 68(4) J. VIROLOGY 2731-34 (1994); Makoto Hijikata et al., 90 PROC. NATL. ACAD. SCI. USA 10773-77 (1993).

The non-structural proteins NS3, NS4A, NS4B, NS5A and NS5B form the viral replication complex and are released from the polyprotein. A zinc-dependent protease associated with NS2 and the N-terminus of NS3 is responsible for cleavage between NS2 and NS3. Arash Grakoui et al., 67(3) J. VIROLOGY 1385-95 (1993); Makoto Hijikata, 90 PROC. NATL. ACAD. SCI. USA 10773-77 (1993).

A distinct serine protease located in the N-terminal domain of NS3 is responsible for proteolytic cleavages at the NS3/NS4A, NS4A/NS413, NS4B/NS5A and NS5A/NS5B junctions. Ralf Bartenschlager et al., 67(7) J. VIROLOGY 3835-44 (1993); Arash Grakoui et al., 90 PROC. NATL. ACAD. SCI. USA 10583-87 (1993); Licia Tomei et al., 67(7) J. VIROLOGY 4017-26 (1993); Arash Grakoui et al., 67(5) J. VIROLOGY 2832-43 (1993). RNA stimulated NTPase and helicase activities are located in the C-terminal domain of NS3. The NS3/4A protease is a validated clinical target for HCV antiviral therapy. Daniel Lamarre et al., 426 Nature 186-89 (2003).

NS4A provides a cofactor for NS3 protease activity. Cristina Fulla et al., 68(6) J. VIROLOGY 3753-60 (1994); De Francesco et al., U.S. Pat. No. 5,739,002.

NS5A is a highly phosphorylated protein conferring interferon resistance. J.-M. Pawlotsky, 6(Suppl. 1) J. VIRAL HEPATITIS 47-48 (1999).

NS5B provides an RNA-dependent RNA polymerase. De Francesco et al., International Publication No. WO 96/37619, published Nov. 28, 1996; Sven-Erik Behrens et al., 15(1) EMBOJ 12-22 (1996); Volker Lohmann et al., 249 VIROLOGY 108-18 (1998). Soluble RNA-dependent RNA polymerase can be produced by a 21 amino acid truncation at the C terminus. Tatsuya Yamashita et al., 273(25) J. BIO. CHEM. 15479-86 (1998); Eric Ferrari et al., 73(2) J. VIROLOGY 1649-54 (1999).

Different genotypes and quasispecies of HCV have been identified. Patrizia Farci et al., 20(1) SEMINARS IN LIVER DISEASE 103-24 (2000); Hiroaki Okamoto et al., 188(1) VIROLOGY 331-41 (1992). There are genotype-specific differences in response to the present preferred treatment of pegelyated alpha interferon administered with ribivarin. Thierry Poynard et al., 352 LANCET 1426-32 (1998); M. P. Manns, 358 LANCET 958-65 (2001); John G. McHutchinson et al., 339(21) NEW ENG. J. MED. 1485-92 (1998); Stephanos J. Hadziyannis et al., ANNALS INTERNAL MED. 346-55 (2004). Genetic variation also results in differential responses to antiviral inhibitors, and is a concern for the development of HCV therapy. Steven W. Ludmerer et. al, 49(5) ANTIMICROBIAL AGENTS & CHEMOTHERAPY 2059-69 (2005).

A cell-based assay for monitoring hepatitis C virus NS3/4A protease activity expressed from a stably maintained con1 replicon in mammalian cells such as those from the human hepatoma Huh-7 cell line, using secreted alkaline phosphatase (SEAP) reporter, is described by Jin-Ching Lee et al., 316(2) ANAL. BIOCHEM. 162-70 (2003).

A reporter construct for NS3/4A activity using secretory alkaline phosphatase-1 (SEAP-1) reporter, which harbors the NS3/4A cleavage site inserted between the SEAP and a membrane anchor featuring an endoplasmic reticulum retention sequence, is described by Laura Pacini et al., 331 ANAL. BIOCHEM. 46-59 (2004). The reporter served to monitor activity of a specific, stably integrated NS3/4A gene.

Development of broad-spectrum HCV antivirals is challenging due to the high genetic complexity intrinsic to circulating viral populations in chronic HCV infection. HCV is broadly classified into six genotypes and more than 100 sub-types. Viruses classified within the same subtype can differ by as much as 5% of the nucleotide sequence, and differences between genotypes range as high as 30%. The complex quasispecies population of variants observed within circulating species increases the likelihood for the pre-existence of variants resistant to a particular drug regimen and which may emerge during treatment. Inhibitors of a specific genetic isolate may have reduced or no potency against diverse clinical isolates and will be poor candidates for antiviral development. Donald J. Graham et al., 69(1) ANTIVIRAL RESEARCH 24-30 (2006).

### SUMMARY OF THE INVENTION

The invention is a method for measuring protease activity from transient expression of a novel HCV NS3/4A sequence, comprising:
a) obtaining said sequence and cloning said sequence into a mammalian expression vector;
b) transiently transfecting a mammalian cell with said vector, wherein said mammalian cell comprises a reporter construct, wherein said reporter construct comprises a nucleotide sequence encoding a HCV NS3/4A cleavage site fused to a detectable label;
c) measuring signal production from said label resulting from cleavage at said cleavage site; and
d) measuring effects on signal production by addition of a test compound.

The method is a transient assay that evaluates activity of diverse NS3/4A clinical or experimental isolates and their sensitivity to protease inhibitors. In one embodiment, individual NS3/4A sequences, such as clinical patient sequences or experimental sequences, can be co-transfected along with a secreted alkaline phosphate reporter construct. Alkaline phosphatase is secreted into the medium only upon release from an ER-tethering transmembrane domain through cleavage of an intervening NS3/4A cleavage site. The method enables measurement of changes in response to a drug regimen due to the rise of resistant variants of the NS3/4A protease.

### DETAILED DESCRIPTION OF THE INVENTION

In one embodiment of the invention, the reporter is secreted alkaline phosphatase fused to the transmembrane domain of the beta adrenergic receptor and separated by said cleavage site. In a further embodiment of the invention, the cleavage site can be the NS5A/B protease cleavage site ATVSEEASEDVVCCSMSYTWTGAL (SEQ ID NO. 1) or another site recognized by NS3/4A protease. In a further embodiment of the invention, the secreted alkaline phosphatase remains intracellular unless released from a tethering domain by cleavage induced by NS3/4A serine protease.

The method can be performed to evaluate the ability of a test compound to inhibit cleavage, and can also be performed to individually evaluate the ability of two or more different compounds to inhibit cleavage.

A HCV NS3/4A sequence is a sequence obtained, for example, from the serum sample of a clinical patient. A HCV NS3/4A sequence can also be obtained experimentally. For example, the experimental NS3/4A sequence may include mutant sequences from chimp studies, in vitro selection, or other derivative sequences generated by experimental protocols, including those where it is useful to isolate emerging mutant clones.

Suitable mammalian cells include mammalian cells Hela, Hek or Huh-7 cells, or derivative cells, or other adherent transfectable mammalian cell lines. A particular example of a suitable cell is a human hepatoma Huh-7 cell.

Suitable reporter constructs which comprise a nucleotide sequence encoding a HCV NS3/4A cleavage site fused to a detectable label are those which allow for measurement of signal production from the label resulting from cleavage at the cleavage site. Such reporter constructs are, for example, those encoding secreted alkaline phosphatase fused to the transmembrane domain of the beta-adrenergic receptor and separated by a polypeptide comprising the NS5A/B protease cleavage site ATVSEEASEDVVCCSMSYTWTGAL. Other reporters producing a chemiluminescent signal such as luciferase may be used. Alternative constructs may include alternative artificial or natural cleavage sites such as those described in Arash Grakoui et al., 67(5) J. VIROLOGY 2832-43 (1993).

Suitable mammalian expression vectors include any standard mammalian expression vector that can be used to introduce foreign genetic material into a mammalian host cell in order to replicate and amplify the foreign DNA sequences.

Transiently transfected cells are those which are transfected with a sequence that is not maintained in the cell. In transient transfection, the introduced gene can be lost from the cell at any time depending on environmental factors.

Techniques for isolating HCV NS3/4A sequences from clinical samples are those which are generally known to persons skilled in the art, such as those described in Xiao Tong et al., 45(5) BIOCHEM. 1353-61 (2006).

In a more specific embodiment, the invention is a method for rapidly identifying active NS3/4A serine protease sequences from HCV-infected serum samples, and determining differences among the sequences in sensitivity to a test compound protease inhibitor, which comprises:
a) rescuing NS3/4A serine protease gene sequences from the serum samples;
b) generating protease cDNAs by reverse transcriptase-polymerase chain reaction;
c) cloning the cDNAs into mammalian expression vectors;
d) transiently transfecting mammalian cells with each cDNA and a corresponding reporter construct encoding secreted alkaline phosphatase fused to the transmembrane domain of the beta-adrenergic receptor and separated by the NS5A/B protease cleavage site ATVSEEASEDVVCCSMSYTWTGAL, which secreted alkaline phosphatase remains intracellular unless released from a tethering domain by cleavage induced by NS3/4A serine protease;
e) assessing the activity of the test compound protease inhibitor by measuring secreted alkaline phosphatase activity from aliquots of the cell-culture supernatant after transfection.
   The invention will monitor changes in susceptibility of NS3/4A protease to an inhibitor due to mutations or genetic variability which arises in NS3/4A protease in a clinical trial of an investigative compound.

The invention is also a transiently transfected mammalian cell comprising NS3/4A serine protease cDNA and a reporter construct encoding secreted alkaline phosphatase fused to the transmembrane domain of the beta-adrenergic receptor and separated by the NS5A/B protease cleavage site ATVSEEASEDVVCCSMSYTWTGAL, in which secreted alkaline phosphatase remains intracellular unless released from a tethering domain by cleavage induced by NS3/4A serine protease, wherein the NS3/4A serine protease cDNA corresponds to HCV genotypes selected from the group consisting of 1a, 1b, 2a, 2b, and 3a or other HCV genotypes. In one embodiment, the NS3/4A serine protease cDNA corresponds to HCV genotype 1a. In another embodiment, the NS3/4A serine protease cDNA corresponds to HCV genotype 1b. In another embodiment, the NS3/4A serine protease cDNA corresponds to HCV genotype 2a. In another embodiment, the NS3/4A serine protease cDNA corresponds to HCV genotype 2b. In another embodiment, the NS3/4A serine protease cDNA corresponds to HCV genotype 3a.

The assay can be performed in a 96-well format suitable for compound titrations and detection in an automated microtiter plate luminometer. It can assist the rapid identification of active sequences among the entire population derived by reverse transcriptase-polymerase chain reaction of clinical specimens, and measure differences in response to inhibitors of interest among clinically-derived sequences. The assay should facilitate analysis of resistance analysis from in vitro selections or among patient populations undergoing drug regimens.

The invention is a transient assay to quickly identify active NS3/4A sequences for further biological and biochemical characterization. Sequences derived from clinical specimens may be inactive for any number of reasons including pre-mature termination, frameshift, or enzymatically inactivating mutations. Isolates that are inactive due to frameshift or premature termination can be identified by sequencing, but sequencing multiple isolates is laborious and time consuming. Furthermore, mutations which impair activity or stability likely require phenotypic evaluation. The assay of the invention enables rapid detection of enzymatically active NS3/4AA sequences, which will facilitate the generation of diverse NS3/4A isolate panels for evaluation of broad-spectrum protease inhibitors.

Cell-culture selection and biochemical analysis have defined a panel of mutations within NS3/4A conferring reduced susceptibility. Some of these mutations have also been identified among residual or rebound viral populations in early clinical studies of these inhibitors. Similar correlations between laboratory and clinical resistance have been described for HIV protease inhibitors. While cell-culture selections provide guidance in understanding clinical resistance, the genetic context of the resistance mutation may influence the degree of reduced susceptibility. The present invention enables a direct analysis of clinical isolates for differences in drug susceptibility, and should complement existing methods such as site-directed mutagenesis of a master clone to enhance evaluation of NS3/4A inhibitors compounds undergoing clinical evaluation.

Unless particular terms are mutually exclusive, reference to “or” indicates either or both possibilities. Occasionally phrases such as “and/or” are used to highlight either or both possibilities.

Reference to “comprises” is open-ended allowing for additional elements or steps. Occasionally phrases such as “one or more” are used with or without “comprises” to highlight the possibility of additional elements or steps.

Unless explicitly stated reference to terms such as “a” or “an” is not limited to one. For example, “a cell” does not exclude “cells”. Occasionally phrases such as one or more are used to highlight the presence of a plurality.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### Reporter Construct for Detection of NS3/4A Activity.

The reporter construct, described by Laura Pacini et al., 331 ANAL. BIOCHEM. 46-59 (2004), encodes the secreted alkaline phosphatase gene followed by three transmembrane spanning domains of the human β-2 adrenergic receptor. The transmembrane domains are separated from the secreted alkaline phosphatase domain by the NS5A/B protease cleavage site of the genotype 1b BK strain, ATVSEEASEDVVCCSMSYTWTGAL. Secreted alkaline phosphatase remains tethered to the endoplasmic reticulum unless cleavage at the NS5A/B site occurs.

### Transient Expression of NS3/4A Compound Titration and Analysis.

The night before transfection, Huh-7 cells are seeded at a density of 6000 cells/well in a 96-well flat-bottom cluster plate in a volume of 150 μl D-MEM complete medium. The following morning, the media is removed by aspiration and replaced with 93 μl of D-MEM without serum and antibiotics, but supplemented with glutamine and non-essential amino acids. Separately, per well 4.775 μl of Optimem I media (Invitrogen) and 0.225 ml of Fugene 6 (Roche) are mixed and incubated for 5 minutes at room temperature. To this, 30 ng of reporter DNA and 20 ng of NS3/4A DNA are added. The sample is vortexed, incubated at room temperature for 15 minutes, and added to the well. For compound titrations, 2 μl of 3-fold serial dilutions of compound in 50% DMSO are added to the wells about 15 minutes following transfection of DNA, thus adjusting the final DMSO concentration to 1.0%.

The following day, 50 μl of supernatant is removed, placed into a Microlite™ 96-well flat bottom plate (Thermo Labsystems, Franklin, Mass.), assayed with the Tropix Phospha-Light™ System (Applied Biosystems, Bedford, Mass.) according to manufacturer's instructions, and analyzed on a MLX Microtiter Plate Luminometer (Dynex Technology). Compound titrations were performed in triplicate using an 8-point series of three-fold compound dilutions. For each titration, the maximal SEAP signal was established with a no drug DMSO control, while background SEAP activity was established by substituting an equivalent amount of pcDNA in place of the protease vector in one triplicate set per titration. Data was fit to a modified Levenberg-Marquardt non-linear regression fitting algorithm using the Visual Numerics IMSL C library (Visual Numbers, San Ramon, Calif.). Compound titrations were performed in triplicate using an 8-point series of three-fold compound dilutions. Data was fit to a modified Levenberg-Marquardt non-linear regression fitting algorithm using the Visual Numerics IMSL C library (Visual Numbers, San Ramon, Calif.).

### NS3/4A Expression Constructs.

The genotype 1b NS3/4A expression construct spanned nucleotides 3420-5474 of the HCV genome con1 strain, corresponding to amino acids 1027-1713 of the corresponding polyprotein. ATG and UGA triplets were engineered into the 5′ and 3′ ends respectively. Novel protease constructs were designed similarly by amino acid alignment.

### EXAMPLE

### (3R,5S,8S)-8-tert-butyl-N-((1R,2S)-1-{[(cyclopropylsulfanyl)amino]carbonyl}-2-vinylcyclopropyl)-18-methoxy-7,10-dioxo-22-phenyl-2,11-dioxa-6,9,21-triazatetracyclo[15.6.2.1^{3,6}.0^{20,24}]hexacosa-1(23),17,19,21,24-pentaene-5-carboxamide

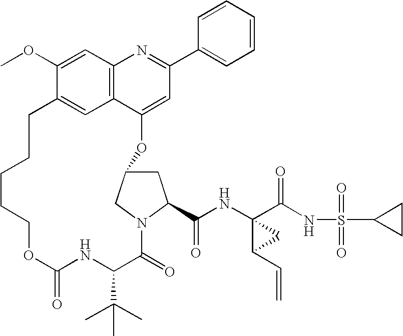

The title compound, also referred to as Compound A, may be prepared by the procedures disclosed in International Patent Application Publication No. WO 2006/119061.

### Compound A Sensitivity Evaluation

The activity of Compound A against a NS3/4A genotype panel was extensively characterized and is shown in Table 1. The compound is extremely potent against genotype 1, retains potency against genotype 2, but loses significant potency against genotype 3. All values are averaged over a minimum of 3 independent titrations, each of which was itself perform in triplicate.

TABLE 1
Activity of Compound A against an NS3/4A genotype panel

| Genotype | EC50 (nM) |
|---|---|
| 1b (con1) | 0.9 +/− 0.4 |
| 1a (H77) | 2.7 +/− 2.2 |
| 2a (JFH1) | 13.6 +/− 8.4 |
| 2b (cs8) | 26.4 +/− 8.6 |
| 3a (ps21) | 890 +/− 220 |

To examine potential shifts in Compound A potency against the genetic variability encountered among clinical sequences, multiple sequences of genotype 1b, 2b, and 3a were isolated from infected patient samples, cloned, and tested for Compound A sensitivity (Tables 2, 3, and 4). 30-fold, 15-fold, and 3-fold differences in Compound A sensitivity were observed among the genotype 1b, 2b and 3a clinical isolates, respectively. Although there was less difference in response among the genotype 3a sequences, all were 10²-10³ less sensitive than genotype 1 and 2 sequences.

TABLE 2
Efficacy of Compound A against clinical
genotype 1b NS3/4A sequences

| Genotype 1b | |
|---|---|
| NS3/4A sequences | EC50 (nM) |
| con1 | 0.9 +/− 0.4 |
| ps20 | 0.9 +/− 0.5 |
| ps30 | 3.5 +/− 0.6 |
| ps31 | 0.9 +/− 0.3 |
| ps32 | 0.1 +/− 0.01 |
| ps33 | 4.0 +/− 0.8 |

TABLE 3
Efficacy of Compound A against clinical
genotype 2b NS3/4A sequences

| Genotype 2b | |
|---|---|
| NS3/4A sequences | EC50 (nM) |
| cs8 | 26.4 +/− 8.6 |
| ps9 | 7.3 +/− 4.1 |
| ps11 | 17.6 +/− 3.1 |
| ps12 | 13.4 +/− 4.0 |
| ps13 | 1.8 +/− 0.8 |
| ps14 | 26.5 +/− 10.1 |

TABLE 4
Efficacy of Compound A against clinical
genotype 3a NS3/4A sequences

| Genotype 3a | |
|---|---|
| NS3/4A sequences | EC50 (nM) |
| ps15 | 360 +/− 150 |
| ps17 | 220 +/− 140 |
| ps21 | 890 +/− 220 |
| ps22 | 410 +/− 240 |
| ps23 | 260 +/− 110 |
| ps24 | 440 +/− 150 |
| cs160 | 420 +/− 200 |

Resistance selections against several different protease inhibitors defined mutations at residues A¹⁵⁶ or residue D¹⁶⁸ as commonly conferring resistance. To investigate whether similar mutations directly affect Compound A susceptibility, A156S, A156T, and D168V substitutions were engineered into the genotype 1b con1 clone. Table 5 shows that the A156S substitution had little effect on sensitivity to Compound A. The A156T and D168V mutations conferred significant loss of Compound A potency.

TABLE 5
Effect of common resistance mutations
on sensitivity to Compound A

| Genotype 1b | |
|---|---|
| NS3/4A and substitutions | EC50 (nM) |
| con1 | 0.9 +/− 0.4 |
| con1 A156S | 7.7 +/− 6.2 |
| con1 A156T | 950 +/− 230 |
| con1 D168V | 270 +/− 100 |

Rapid Identification of Active NS3/4A Sequences from Clinical Samples.

An NS3/4A cDNA pool derived from an HCV-infected plasma sample may encode many inactive sequences due either to the copying inactive variants or copied inaccurately by the procedures used to generate the cDNA. We used the transient assay to identify active sequences within a cDNA pool generated from a genotype 2b infected plasma sample (ps9) prior to a more thorough biological/biochemical characterization. A cDNA pool was generated using genotype 2b specific primers. Mini-prep DNA from single clones were transfected to evaluate NS3/4A activity. A dozen clones were randomly chosen for analysis. While 11 of 12 clones encoded full-length inserts by restriction mapping, only six were active in the NS3/4A transient assay. These results, shown in Table 6, enabled selection of active clones for biochemical characterization.

TABLE 6
Identification of active NS3/4A sequences

| ps9# | NS3/4A insert | NS3/4A (SEAP) Activity |
|---|---|---|
| 1 | + | − |
| 2 | + | + |
| 3 | + | − |
| 4 | + | + |
| 5 | + | − |
| 6 | + | + |
| 7 | + | − |
| 8 | + | + |
| 9 | + | + |
| 10 | + | − |
| 11 | + | + |
| 12 | + | − |

Other embodiments are within the following claims. While several embodiments have been shown and described, various modifications may be made without departing from the spirit and scope of the present invention.

What is claimed is:

1. A method for measuring HCV NS3/4A activity from transient expression of novel HCV NS3/4A sequences, comprising:
a) obtaining said sequence and cloning said sequence into a mammalian expression vector;
b) transiently transfecting a mammalian cell with said vector, wherein said mammalian cell comprises a reporter construct, wherein said reporter construct comprises a nucleotide sequence encoding a HCV NS3/4A cleavage site fused to a detectable label;
c) measuring signal production from said label resulting from cleavage at said cleavage site; and
d) measuring effects on signal production by addition of a test compound.

2. The method of claim 1, wherein said reporter is secreted alkaline phosphatase fused to the transmembrane domain of the beta adrenergic receptor and separate by said cleavage site.

3. The method of claim 1, wherein said HCV NS3/4A sequence is obtained from a serum sample or is an experimental sequence.

4. The method of claim 2, wherein said method is performed to evaluate the ability of the test compound to inhibit cleavage.

5. The method of claim 2, wherein said method is performed to individually evaluate the ability of two or more different test compounds to inhibit cleavage.

6. The method of claim 2, wherein said cleavage site is the NS5A/B protease cleavage site SEQ ID NO. 1.

7. The method of claim 2, wherein said cleavage site is recognized and cleaved by NS3/4A protease.

8. The method of claim 2, wherein said secreted alkaline phosphatase remains intracellular unless released from a tethering domain by cleavage induced by NS3/4A serine protease.
